(19) United States
(12) Patent Application Publication
(10) Pub. No.: US 2008/0058507 A1
(43) Pub. Date: Mar. 6, 2008

(54) Method For The Removal Of Aggregate Proteins From Recombinant Samples Using Ion Exchange Chromatography

(76) Inventors: Hui Liu, Houston, TX (US); Ke Tang, Worcester, MA (US)

Correspondence Address: Nikolaos C. George;JONES DAY, 222 E. 41st. Street, New York, NY, 10017, US

(21) Appl. No.: 10/589,068

(22) PCT Filed: Feb. 10, 2005
(86) PCT No.: PCT/US05/04729

§ 371 Date: Jul. 19, 2007

Related U.S. Application Data
(60) Provisional application No. 60/521,047, filed on Feb. 11, 2004.
(60) Provisional application No. 60/568,374, filed on May 5, 2004.

(51) Int. Cl. C07K 1/18

(52) U.S. Cl. 530/416

(57) ABSTRACT

The present invention relates to processes for the removal of unwanted protein aggregates from antibody preparations. One process involves removal when the aggregate and antibody are very close in pI value (“Bind-Elute” process). Another process involves the removal when the aggregate and antibody are very close in net electric charge and retention time on ion exchange resin (“Bind-Washout” process). Using either process, at least 90% of the antibody is recovered and 70-90% of the aggregate is removed.

### FIELD OF THE INVENTION

The present invention relates to processes for the removal of unwanted protein aggregates from antibody preparations. One process involves removal when the aggregate and antibody are very close in pI value (“Bind-Elute” process). Another process involves the removal when the aggregate and antibody are very close in net electric charge and retention time on ion exchange resin (“Bind-Washout” process).

### BACKGROUND

Protein solutions such as immunoglobulins (lgG), including polyclonal and monoclonal antibodies, routinely contain protein aggregates comprising dimers or higher polymers. In order to administer this solution to a patient, it is necessary to first remove these aggregates to avoid a potentially adverse reaction In the patient.

The aggregates present in a biological preparation should be controlled below a certain range (e.g., <1%) in purified antibody preparations for clinical application. Accordingly, it is desirable to provide a process for removing protein aggregates from an antibody preparation on a commercial scale by a filtration process which is fast, economic and selectively removes at least 90% of the aggregates with very little loss of the commercial product.

Previously, as disclosed in e.g., U.S. Pat. No. 6,177,548 and U.S. Pat. No. 6,281,336, the typical method to remove the majority of aggregate from an antibody preparation used ion exchange chromatography by a flow-through process. In this process, the pH was controlled below the protein's pl range and the ionic strength was chosen in such a way that a major portion of the contaminants (e.g., aggregates) in the applied solution bound to the anion exchange resin, whereas substantially no antibody monomer was adsorbed to the anion exchange resin, and the desired antibody product was recovered in the flow through.

As more and more antibodies are developed for therapeutic purposes, it has become necessary to develop a process of quickly determining the pH and salt conditions sufficient to adequately separate aggregates from the antibody of interest. Moreover, many of these aggregates cannot be separated from the antibody monomer of interest by traditional linear gradients, while using a process that is cost efficient and scalable to a level sufficient for commercial manufacturing. Many resins that can be used at small scales are not useful at large scale because of factors such as column volume, back pressure or cost.

The present invention provides a new improved process for quickly determining the conditions necessary and the separation of difficult to remove aggregates from an antibody monomer preparation, wherein the process is scalable to a commercial manufacturing level.

### SUMMARY OF THE INVENTION

The present invention provides a process for aggregate removal involving a “bind-elute” process for efficient removal of aggregates under conditions suitable for manufacturing scale purification of antibodies. In addition, the antibody product to be purified does not require a pre-conditioning step, such as tangential flow filtration, as with some previous methods.

The advantage of the present invention is the ability to purify antibody monomers of interest from an antibody preparation wherein the pi ranges of the the antibodies and the aggregates formed overlap. The pI values of the aggregates formed overlap with the range of pI values for the antibody monomers making it difficult or impossible to separate the two by traditional linear gradient conditions. The present invention provides a process for separation of such aggregates on a chromatography column and such that, when the purification process is scaled up to manufacturing levels, the aggregate can be efficiently removed from the preparation.

The “bind-elute” process for the manufacturing scale purification of antibody monomers from a recombinant antibody sample containing aggregates comprises: choosing a resin suitable for manufacturing level purification; determining a pH value and a salt concentration to be used in the manufacturing level purification such that the antibody monomers and the aggregates bind to the resin; loading the recombinant antibody sample onto the chosen resin; and eluting the antibody monomers from the resin using a step gradient.

Under the selected conditions of the present invention, both the antibody and its aggregate bind to the resin during loading step. Efficient separation of antibody and its aggregate is achieved by applying a step gradient elution program to elute the antibody monomer, leaving the aggregate bound to the anion exchange resin. The present invention achieves a recovery of at least 85% of the desired antibody, preferably 90%, more preferably 95%, with less than 0.1% aggregate remaining.

The present invention also provides a process for aggregate removal involving a “bind-washout” process for efficient removal of aggregates under conditions suitable for manufacturing scale purification of antibodies using a single buffer.

The advantage of the present invention is the ability to purify antibody monomers of interest from an antibody preparation where the aggregates formed are very close in net electric charge and retention time on ion exchange chromatographic resin and the aggregate would not separate from the monomer under normal linear gradient conditions with ion exchange resin. The present invention provides a process for separation of such an aggregate on a chromatography column such that when the purification process is scaled up to manufacturing levels the aggregate is efficiently removed from the preparation.

The “bind-washout” process for the manufacturing scale purification of antibody monomers from a recombinant antibody sample containing aggregates comprises: choosing a resin suitable for manufacturing level purification; determining a pI value for the antibody monomer to be purified; determining a pH value and a salt concentration to be used in the manufacturing level purification based on the pI value, wherein the aggregates bind to the resin and wherein the antibody monomers interact weakly with the resin; and loading the recombinant antibody sample onto the chosen resin and washing the antibody monomers from the resin with a single buffer resulting in manufacturing level purification of the antibody monomers.

Under the selected conditions of the present invention, when following the “bind-washout” process, the recombinant antibody sample is loaded onto a resin and the antibody monomers flow slowly through the resin during the loading step and during the wash step using a single buffer, while the aggregate binds to the resin. In this manufacturing scale process, there is a weak interaction between the antibody and the resin and the monomers are relatively slowly washed out by applying buffer. About 70-80% of aggregate can be removed by this “bind-washout” process. This process is used when, for example, the salt concentration in the starting antibody sample is too high and/or the pH is too low to proceed with the “bind-elute” process described above.

### BRIEF DESCRIPTION OF THE FIGURES

FIG. 1 depicts the antibody concentration and aggregate levels in each antibody fraction versus elution volume for TNX-901 following anion exchange.

FIG. 2 depicts a chromatogram of the Q-SEPHAROSE® run for TNX-901 at pH 9.2 using the “bind-elute” process.

FIG. 3 depicts the antibody concentration and aggregate levels in each antibody fraction versus elution volume for TNX-355 following anion exchange.

FIG. 4 presents the results of TNX-355 aggregate removal using the “bind-washout” process.

FIG. 5 depicts a chromatogram of the Q-SEPHAROSE® run for TNX-355 at pH 8.0 using the “bind-elute” process.

FIG. 6 depicts a chromatogram of the Q-SEPHAROSE® run for TNX-355 at pH 8.2 using the “bind-washout” process.

### DETAILED DESCRIPTION

This invention is not limited to the particular methodology, protocols, cell lines, vectors, and reagents described herein because they may vary. Further, the terminology used herein is for the purpose of describing particular embodiments only and is not intended to limit the scope of the present invention. As used herein and in the appended claims, the singular forms “a, ” “an,” and “the” include plural reference unless the context clearly dictates otherwise, e.g., reference to “an antibody” includes a plurality of such antibodies.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the methods, devices, and materials are described herein.

All patents and publications mentioned herein are incorporated herein by reference to the extent allowed by law for the purpose of describing and disclosing the proteins, enzymes, vectors, host cells, and methodologies reported therein that might be used with the present invention. However, nothing herein is to be construed as an admission that the invention is not entitled to antedate such disclosure by virtue of prior invention.

As is well known in the art, ion exchangers may be based on various materials with respect to the matrix as well as to the attached charged groups. For example, the following matrices may be used, in which the materials mentioned may be more or less cross-linked: agarose based (such as SEPHAROSE Fast Flow® (such as Q-SEPHAROSE FF), and SEPHAROSE High Performance®; cellulose based (such as DEAE SEPHACEL®); silica based and synthetic polymer based, or resins such as SuperQ-650 (from TOSOH BIOSEP) and Macro High Q (from BIO-RAD). For the anion exchange resin, the charged groups which are covalently attached to the matrix may, e.g., be diethylaminoethyl (DEAE), quaternary aminoethyl (QAE), and/or quaternary ammonium (Q). In a preferred embodiment of the present process, the anion exchange resin employed is Q-SEPHAROSE FF®, but other anion exchangers can be used.

Although any of these resins may be used for small scale purification of antibodies, only those resins of sufficient size and lower cost are amenable to manufacturing scale separation. If the size of the resin is too small, there is considerable back pressure generated in the system. In addition, the amount of antibody that can be purified is limited. If the resin is costly to make or purchase, it is not economically feasible/practical for use in large scale purification.

Thus, the resin used in the present invention must be of sufficient size to provide efficient scale-up without being prohibitively expensive. “Manufacturing level purification” means purification of antibodies from a recombinant preparation on a scale sufficient to meet commercial scale production. The resin used in the predetermination step should be the same as that used in the final protocol for manufacturing level purification because one may not easily predict the variation in conditions necessary to separate the aggregates if the resin is changed. A particular resin that is useful in small scale or bench top purification may not be amenable to large scale purification. Such resins useful for the present invention include, e.g., Q-SEPHAROSE FF™. However, the skilled artisan would recognize other anion exchange resins useful for commercial scale production.

The appropriate volume of resin used when packing an ion exchange chromatography column is reflected by the dimensions of the column, i.e. the diameter of the column and the height of the resin, and varies depending on, e.g., the amount of antibody in the applied solution and the binding capacity of the resin used.

Before performing an ion exchange chromatography, the exchange resin may be equilibrated with a buffer. Any of a variety of buffers are suitable for the equilibration of exchange resin, e.g. sodium acetate, sodium phosphate, TRIS(hydroxymethyl) amino-methane, TRIS, phosphate, bis-TRIS, and L-histidine. Persons skilled in the art will appreciate that numerous other buffers may be used for the equilibration as long as the pH and conductivity are about the same as for the applied antibody solution. When performing the “bind-washout” process, the equilibration buffers and the wash buffers are the same. When performing the “bind-elute” process, the elution buffers may be made of one or more buffer substances to control the pH. The salt used is, e.g., a highly soluble salt, such as sodium chloride or potassium phosphate, but any salt may be used that maintains the functionality of the antibody and allows removal of the antibody monomer from the resin.

In performing the “bind-elute” process, the elution of the antibody monomers from the resin may be performed with a substantially non-denaturating buffer having a pH and ionic strength sufficient to efficiently elute the monomeric antibody, thereby recovering an antibody-containing eluate, while leaving the aggregates bound to the resin. In this context, efficient elution means that at least 75%, or at least 80%, or at least 85%, or at least 90%, or at least 95% of the antibody loaded onto the resin is recovered. Only about 1.0%, preferably only 0.5%, most preferably less than 0.1% aggregates remain in the antibody preparation following ion exchange.

The elution is advantageously carried out as a step gradient elution step. In the process of the present invention, the preferred buffer used is TRIS having a pH within the range of the pI value of the antibody monomers.

It is preferred that the salt concentration of the eluting buffer is sufficiently high to displace the antibody monomers from the resin without displacing the aggregates. However, it is contemplated that an increase in pH and a lower salt concentration can be used to elute the antibody monomers from the resin.

When performing the “bind-washout” process, the recovery of the antibody from the resin may be performed with a non-denaturing buffer having a pH and an ionic strength sufficient to enable the monomeric antibody to bind weakly to the resin and then to be displaced from the column during the wash step, thereby recovering an antibody monomer-containing pool, while leaving the majority of aggregate bound to the resin. The buffer used for the equilibration and wash steps is the same.

The present invention allows the effective removal of aggregated antibody from an antibody monomer preparation by Q-SEPHAROSE FAST FLOW® chromatography at pH ranges near, as well as higher than, the pI value of the antibody with varied salt concentrations. When the “bind-elute” process is used after an antibody sample is bound to the resin, the antibody monomer can be eluted by changing the salt concentration and/or pH of the elution buffer through a step gradient elution program, leaving the majority of the aggregates bound to the resin. When the “bind-washout” process is used, due to the heterogeneity of pI values of an antibody, the part(s) of the antibody monomer that has/have higher pI values can flow through the resin, and the part(s) of the antibody monomer that has/have lower pI values can bind weakly on the resin and can be eluted from the resin by a certain volume of equilibration buffer, after an antibody sample is loaded on the resin. The efficiency of aggregate removal by the optimized step gradient elution is generally 80-90% in the “bind-elute” process. About 70-90% of aggregate removal can be achieved in the “bind-washout” process.

The antibody to be purified may be of any isotype, including IgG1, IgG2, IgG3, IgG4, IgM, etc.

### EXAMPLES

### Example 1

### Sample Preparation Prior to Chromatography

A cell culture supernatant containing a desired antibody to be purified may be processed through an immobilized Protein A column, e.g., PROSEP A (Millipore) or MabSelect (Amersham Biosciences). The antibody sample may also be applied to a hydrophobic interaction column (HIC), e.g., Phenyl SEPHAROSE FAST FLOW® (Amersham Biosciences), or cation exchange, e.g, CM-SPEHAROSE FAST FLOW® (Amersham Biosciences). The antibody preparation may be stored under appropriate conditions at this stage. The present process for aggregate removal may also be performed prior to any purification steps.

In this example, an anti-IgE antibody, TNX-901, comprising an IgG1 framework was used. Prior to Q-SEPHAROSE FF® anion exchange chromatography, the monoclonal antibody sample was adjusted to the desired pH for loading the antibody onto a Q-SEPHAROSE FF® column with, e.g., Tris buffer. The salt concentration of the sample was adjusted, based upon the designed loading antibody condition. Sample buffer exchange may be done using a variety of column resins, e.g., SEPHADEX G-25 Fine resin, or by direct conditioning without a column or filtration.

### Example 2

### Determination of Aggregate Concentration

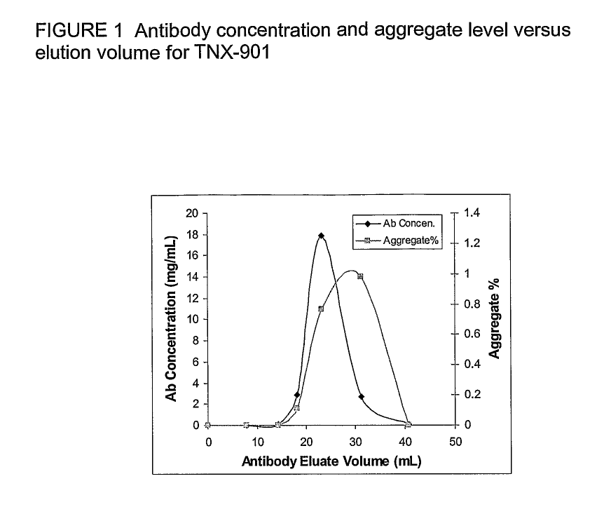

The percentage of the aggregates in each prepared sample was determined, including the fractions collected through sample loading, washing and eluting steps. Superdex 200 HR pre-packed analytical column may be used to determine percent aggregate level. FIG. 1 depicts the antibody concentration and aggregate levels in each antibody fraction versus elution volume for the antibody TNX-901 following anion exchange.

### Example 3

### Determination of Antibody Binding and Elution Conditions for TNX-901

The appropriate salt concentrations used for loading and eluting conditions at the various pH conditions were determined by applying a linear gradient of salt concentration increments under designed pH conditions. The pH range determined for loading the TNX-901 antibody sample was 8.2 to 9.2. At pH 8.2 the antibody monomers and aggregates both bind onto Q-SEPHAROSE FF® resin with loading buffer of 10 mM TRIS.

However, the salt concentrations of the buffers for loading and eluting antibody sample may be simultaneously increased when the loading buffer pH is increased from 8.2 to 9.2 for TNX-901 antibody.

For example, a Q-SEPHAROSE FF® column (1 cm ID×9 cm height) was equilibrated with 10 mM TRIS at pH 8.6. The buffer-adjusted antibody sample prepped according to Example 1 was then loaded onto the column. The antibody was loaded at −17 mg/ml of resin. Under these conditions the antibody monomers and aggregates bound to the column.

The column was washed with 5 column volumes of equilibrating buffer (10 mM TRIS, pH 8.6) after loading the antibody.

The bound antibody was eluted with 15 column volumes of the buffer containing 10 mM TRIS, pH 8.6 using a linear salt gradient from 0to 500 mM. Fractions were collected over the course of the elution phase.

Antibody concentration and aggregate levels were measured in each antibody fraction. The salt concentration of each antibody fraction was also measured by determining its osmolarity. The results are shown in Table 1.

TABLE 1
Antibody fractions collected from TNX-901 Q run at pH 8.6
with a linear salt gradient.

| | Cummulative | Ab Conc. | | Salt Conc. |
|---|---|---|---|---|
| Fraction # | Elution Volume | (mg/ml) | Aggregate (%) | (mM) |
| 1 | 7.8 | 0.02 | 0.0* | 65 |
| 2 | 14.3 | 0.06 | 0.0* | 132 |
| 3 | 18.2 | 2.89 | 0.11 | 180 |
| 4 | 23.2 | 17.89 | 0.77 | 218 |
| 5 | 31.2 | 2.72 | 0.90 | 282 |
| 6 | 40.7 | 0.11 | 0.0* | 361 |

*Below the detection level of the assay

From these results, it was determined that the salt concentration in the first antibody elution fraction was −65 mM, and thus, the salt concentration that may be used at pH 8.6 to load antibody sample should be below 60 mM. The salt concentration that could be used to elute antibody with a step gradient would be between 65 mM and 135 mM based on the results shown in Table 1 for fractions 1 and 2.

FIG. 1 shows the antibody concentration and aggregate level in each antibody fraction versus eluate volume. Retention time for aggregates is slightly longer than that for the antibody monomer. As one can observe, the differences in retention times for the antibody monomer and aggregates were not sufficient for adequate separation using a linear gradient elution program. Therefore, using the present invention process, the buffers with various pHs were tested with a step-salt gradient, in order to maximize the separation of TNX-901 antibody monomer from its aggregates. The highest salt concentration for loading antibody sample at each pH condition was determined and the final salt concentration of the elution buffer for eluting antibody monomer at each pH condition was also optimized to achieve the maximal percentage of aggregate removal and the high yield of the antibody recovery.

Aggregate removal by Q-SEPHAROSE FF® resin in the “bind-elute” process was studied over a pH range from 8.2 to 9.2. The amount of antibody loaded was in the range of 15-37 mg/ml of resin, and the amount of aggregate loaded was in the range of 0.1-0.25 mg/ml of resin. Table 2 presents the results of aggregate removal and antibody monomer recovery over this pH range.

TABLE 2
Results of Aggregate Removal From TNX-901 Under Different Loading And Eluting Conditions

| pH of Loading Sample | 8.2 | | 8.4 | | 8.8 | | | | 9.2 | | | |
|---|---|---|---|---|---|---|---|---|---|---|---|---|
| Highest Salt Concentration | 0 | mM | 0 | mM | 30 mM | | | | 60 mM | | | |
| in Sample & Loading Buffers | | | | | | | | | | | | |
| Salt Concentration in | 20 | mM | 40 | mM | 70 | mM | 80 | mM | 100 | mM | 110 | mM |
| Elution Buffer | | | | | | | | | | | | |
| Load Ab Amount/mL Resin | 16.9 | mg | 16.3 | mg | 37.7 | mg | 15.4 | mg | 16.9 | mg | 17.2 | mg |
| Load Aggregate Amount/mL Resin | 0.11 | mg | 0.10 | mg | 0.25 | mg | 0.09 | mg | 0.12 | mg | 0.13 | mg |

| | | | | | | |
|---|---|---|---|---|---|---|
| Ab % in Flow Thru | 6.6% | 0 | 13.4% | 0 | 0% | 0 |
| Ab % in Eluate | 86% | 93% | 86% | 98% | 89% | 95% |
| Initial Aggregate Level (%) in Load | 0.64% | 0.62% | 0.66% | 0.58% | 0.71% | 0.77% |
| Aggregate Level (%) in Eluate | 0.03% | 0.04% | 0.14% | 0.09% | 0* | 0.04% |
| Aggregate Removal (%) | 95% | 94% | 79% | 84% | ~100% | 95% |

* under the detection limit of the process

It was determined from these results that a pH of 9.2 was optimal for the highest aggregate removal, while still recovering the majority of the antibody monomer in the eluate.

### Example 4

### Step Gradient Elution Conditions for TNX-901

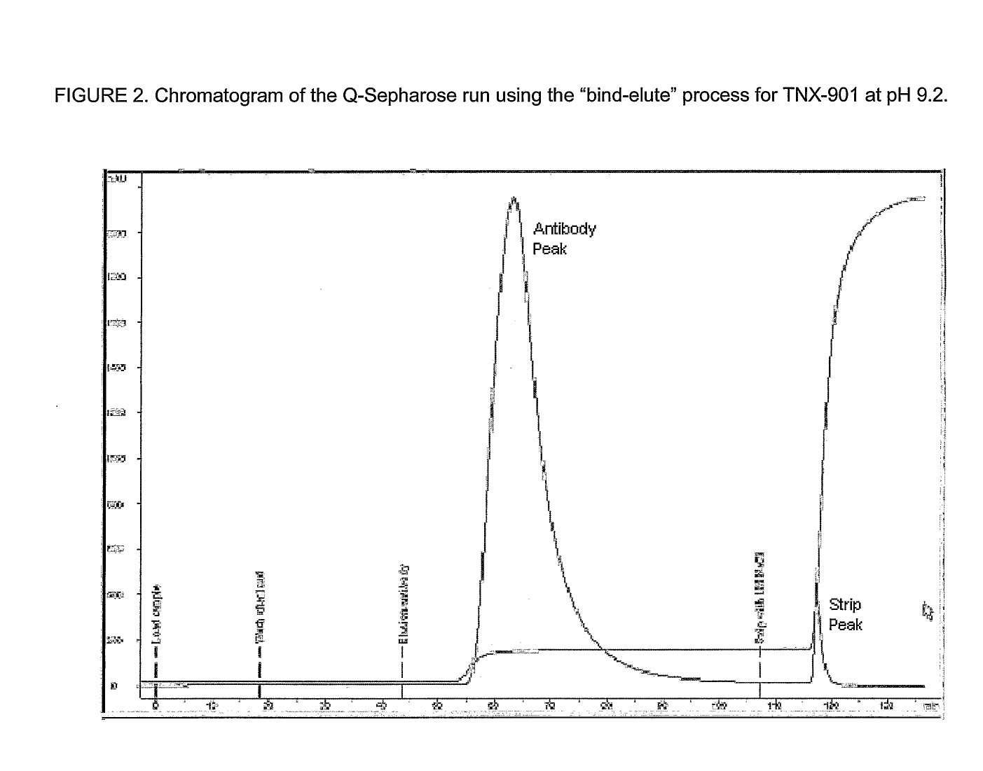

Table 3 shows the operating conditions for the separation of TNX901 on Q-SEPHAROSE FF® at pH 9.2. The Q-SEPHAROSE FF® column was at 1 cm ID×9 cm bed height. The antibody recovery was over 95% under these tested conditions. The antibody peak pool had about 6 column volumes and contained less than 0.1% aggregates. The chromatogram is shown in FIG. 2.

TABLE 3
Chromatographic Conditions for TNX901

| | | Flow Rate | | Volume | |
|---|---|---|---|---|---|
| Step | Solution | cm/hr | ml/min | CV | mL |
| Equilibration | 10 mM Tris, 60 mM | 122 | 1.6 | 8 | 56 |
| | NaCl, pH 9.2 | | | | |
| Loading | Sample | 122 | 1.6 | 4 | 28 |
| Wash | 10 mM Tris, 60 mM | 122 | 1.6 | 6 | 42 |
| | NaCl, pH 9.2 10 mM | | | | |
| | Tris, 110 mM NaCl, pH | | | | |
| Elution | 9.2 | 122 | 1.6 | 15 | 105 |
| Strip | 1 M NaCl | 122 | 1.6 | 7 | 49 |
| Regeneration | 1 M NaCl/1 N NaOH | 76 | 1.0 | 5 | 35 |

### Example 5

### “Bind-Elute” and “Bind-Washout” Conditions for TNX-355 Antibody

In this example, a different monoclonal antibody, TNX-355, was tested having an IgG4 framework. The appropriate salt concentrations used for loading and eluting conditions at the various pH conditions were determined by applying linear gradient of salt concentration increment at the designed pH conditions. The pH range determined for loading TNX-355 antibody sample was 6.5 to 8.2. At the antibody loading condition of low conductivity, most of the antibody and its aggregates bind to the Q-SEPHAROSE FF® resin.

For example, a Q-SEPHAROSE FF® column (1 cm ID×11 cm height) was equilibrated with 10 mM Tris, pH 8.0, and 25 mM NaCl. The buffer-adjusted antibody sample was then loaded onto the column. The buffer-adjusted antibody sample prepped according to Example 1 was then loaded onto the column at −11 mg/ml of resin. Under these conditions the antibody and its aggregates bound to the column.

The column was washed with 6 column volumes of equilibrating buffer (10 mM Tris, 25 mM NaCl, pH 8.0) after loading antibody.

The bound antibody was eluted with 5 column volumes of a linear gradient from 25 mM NaCl to 250 mM NaCl at pH 8.0, and then with 5 column volumes of 250 mM NaCl at pH 8.0. Fractions were collected over the course of the elution phase.

Antibody concentration and aggregate levels were measured in each antibody fraction. The salt concentration of each antibody fraction was also determined by measuring the osmolarity. The results are shown in Table 4.

TABLE 4
Antibody fractions collected from TNX-355 Q
run at pH 8.0 with a linear gradient.

| | | Cummulative | [Ab] | Aggregate | |
|---|---|---|---|---|---|
| Fraction# | OD280 | Elution Volume | mg/ml | % | mOsm/kg |
| 1 | 0.013 | 5.7 | 0.01 | 0.0* | 83 |
| 2 | 0.409 | 10.9 | 0.29 | 0.0* | 127 |
| 3 | 6.25 | 16.4 | 4.46 | 0.10 | 179 |
| 4 | 8.07 | 23 | 5.76 | 0.36 | 238 |
| 5 | 3.04 | 29.8 | 2.17 | 0.62 | 306 |
| 6 | 0.423 | 35.7 | 0.30 | 0.63 | 369 |
| 7 | 0.119 | 42.5 | 0.09 | | 429 |

*Below detection limit.

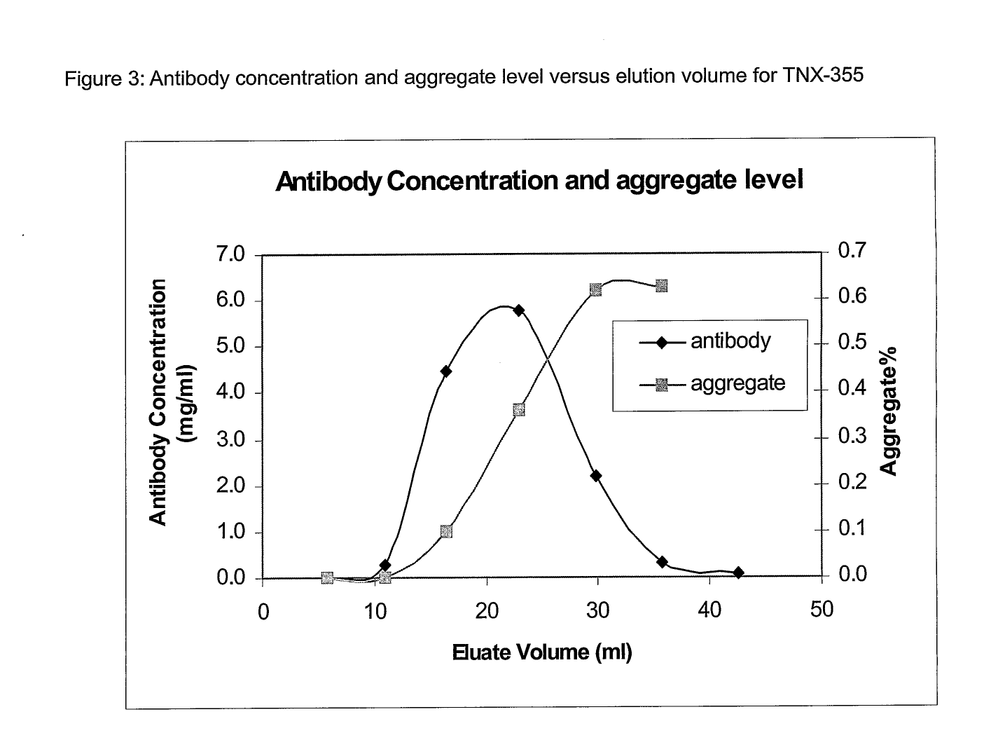

As seen in the results from this table, the salt concentration may be between ˜40 mM (80 mOsmo/kg) and ˜90 mM (180 mOsmo/kg) based on the osmolarity of fractions 1-3 and the point where aggregate begins to elute from the column. FIG. 3 shows the antibody concentration and aggregate levels in each fraction versus elution volume.

At pH 8.0, which was 0.8 pH units higher than the upper limit of TNX-355 antibody pi range, both the antibody and its aggregates bound tightly to the Q resin. By gradually increasing the salt concentration in the elution buffer, the antibody monomer and aggregates were eluted from the Q column at different retention times. A small difference in retention time between the antibody monomer and its aggregates was also observed. An elution step with the optimized pH and salt concentration, which is close to the second fraction condition, in elution buffer can be used to elute the antibody monomer. The Q-SEPHAROSE FF® columns used for this study were at 1 cm ID with 11-20 cm bed height.

Table 5 presents the results under loading and eluting conditions with working pHs at 7.8 and 8.0.

TABLE 5
Results of TNX-355 aggregate removal under different eluting conditions

| | pH of loading sample | | | | | | | | | | | | | |
|---|---|---|---|---|---|---|---|---|---|---|---|---|---|---|
| | 7.8 | | | | 8.0 | | | | | | | | | |
| Run # at designed pH | 1 | | 2 | | 1 | | 2 | | 3 | | 4 | | 5 | |
| Column bed height | 12 | cm | 12 | cm | 11 | cm | 11 | cm | 11 | cm | 20 | cm | 20 | cm |
| Salt concentration in | 25 | mM | 25 | mM | 25 | mM | 25 | mM | 25 | mM | 25 | mM | 25 | mM |
| loading buffer | | | | | | | | | | | | | | |
| Salt concentration in | 75 | mM | 85 | mM | 75 | mM | 85 | mM | 85 | mM | 85 | mM | 90 | mM |
| elution buffer | | | | | | | | | | | | | | |
| Load Ab amount/ml | 17.8 | mg | 18.3 | mg | 9.4 | mg | 20.3 | mg | 20.6 | mg | 20.0 | mg | 19.7 | mg |
| resin | | | | | | | | | | | | | | |
| Load Aggregate | 0.49 | mg | 0.49 | mg | 0.13 | mg | 0.45 | mg | 0.71 | mg | 0.55 | mg | 0.58 | mg |
| amount/ml resin | | | | | | | | | | | | | | |

| | | | | | | | |
|---|---|---|---|---|---|---|---|
| Initial aggregate level | 2.78% | 2.68% | 1.35% | 2.22% | 3.46% | 2.73% | 2.96% |
| (%) in load | | | | | | | |
| Aggregate level (%) in | 0.26% | 0.39% | <0.05% | 0.17% | 0.18% | 0.15% | 0.15% |
| eluate | | | | | | | |
| Aggregate removal (%) | 91% | 74% | >96% | 92% | 95% | 95% | 95% |
| Antibody recovery (%) | 90% | 93% | 89.20% | 96% | 93% | 88% | 89% |

The salt concentration in loading buffer was kept at 25 mM sodium chloride with 10 mM Tris for every run. The amount of antibody loaded was in the range of 9-20 mg/ml of resin, and the amount of aggregate was in the range of 0.13-0.58 mg/ml of resin. The antibody recovery was over 88% under these tested conditions. The antibody peak pool had about 6-7 column volumes and contained less than 0.4% of aggregates.

The results in Table 5 show also that the aggregate removal on Q-SEPHAROSE® resin is more effective at pH 8.0 than 7.8 under the same salt concentration for eluting the antibody. Since the pi range for the TNX-355 antibody is between pH 6.4 and 7.2, using a higher chromatographic pH that is above the pi range will result in the better separation of the antibody from its aggregate.

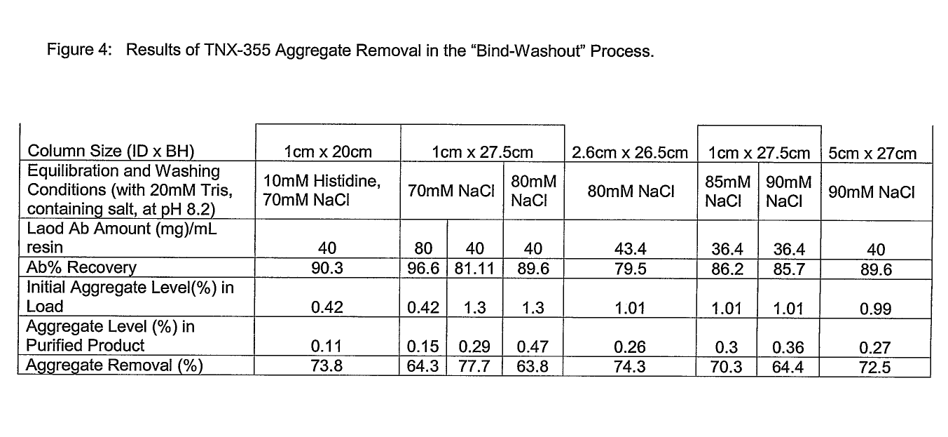

Based on the results shown in Table 4, selection of salt concentration between the fractions 1-3 can be applied in equilibration and washing buffer in the “bind-washout” process. FIG. 4 shows the results of chromatographic runs at selected loading and washing conditions. The loading and washing salt concentrations of 70-90 mM sodium chloride, which were between the fractions of 2-3 listed in Table 4, were tested at pH 8.2. More than 60% of aggregate can be removed and over 80% of the antibody can be recovered. The aggregate level in the purified product was less than 0.5%.

### Example 6

### Step Gradient Elution Conditions Using the “Bind-Elute” Process for TNX-355

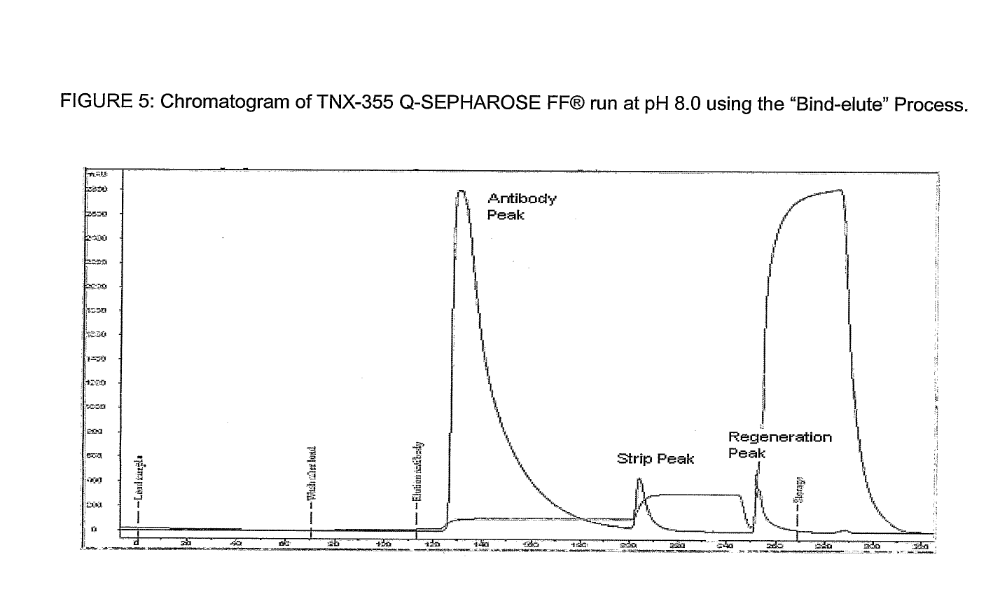

TNX-355 antibody aggregate removal by Q-SEPHAROSE FF® resin using the “bind-elute” process was studied at a pH above 7.5. The process from an example of the Q-SEPHAROSE FF® run at pH 8.0 is presented in Table 7, and its chromatogram is shown in FIG. 5.

TABLE 7
Chromatographic operation conditions for TNX355 using the
“bind-elute” process

| | | | | Number of |
|---|---|---|---|---|
| | | Flow Rate | | Column |
| Step | Solution | cm/hr | ml/min | Volume |
| Equilibration | 10 mM Tris, 25 mM NaCl, | 122 | 1.6 | 8 |
| | pH 8.0 | | | |
| Loading | Sample | 122 | 1.6 | |
| Wash | 10 mM Tris, 25 mM NaCl, | 122 | 1.6 | 5 |
| | pH 8.0 | | | |
| Elution | 10 mM Tris, 85 mM NaCl, | 122 | 1.6 | 9 |
| | pH 8.0 | | | |
| Strip | 10 mM Tris, 250 mM NaCl, | 122 | 1.6 | 5 |
| | pH 8.0 | | | |
| Regeneration | 1M NaCl/1N NaOH | 122 | 1.6 | 4 |

### Example 7

### Bind-Washout Conditions for TNX-355

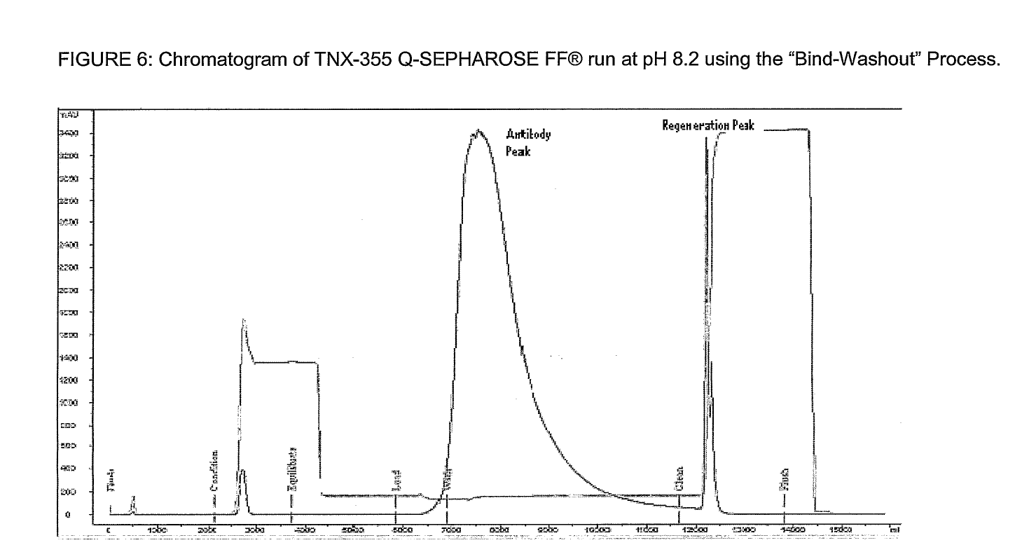

TNX-355 antibody aggregate removal by Q-SEPHAROSE FF® resin using the “bind-washout” process was studied at a pH 8.2. The process from an example of the Q-SEPHAROSE FF® run with a column of 5 cm internal diameter and 27 cm bed height is presented in Table 8, and its chromatogram is shown in FIG. 6.

TABLE 8
Chromatographic operation conditions for TNX355 in
“bind-washout” process

| | | | | Number of |
|---|---|---|---|---|
| | | Flow Rate | | Column |
| Step | Solution | cm/hr | ml/min | Volume |
| Condition | 20 mM Tris, 1M NaCl, | 148.5 | 48.6 | 3 |
| | pH 8.2 | | | |
| Equilibration | 20 mM Tris, 90 mM NaCl, | 148.5 | 48.6 | 4 |
| | pH 8.2 | | | |
| Loading | Sample conditioned to 20 mM | 97.2 | 31.8 | 2 |
| | Tris, 10 mM Histidine, | | | |
| | 70 mM NaCl, pH 8.2 | | | |
| Wash | 20 mM Tris, 90 mM NaCl, | 97.2 | 31.8 | 9 |
| | pH 8.2 | | | |
| Regeneration | 1M NaCl/1N NaOH | 148.2 | 48.6 | 4 |

The preceding description has been presented only to illustrate and describe embodiments of the invention. It is not intended to be exhaustive or to limit the invention to any precise form disclosed. Many modifications and variations are possible in light of the above teaching. For example, although the process as outlined above describes in detail the process for monoclonal antibodies TNX901 and TNX355, the process as described may be readily modified to suit any monoclonal antibody in any given recombinant antibody product.

We claim:

1. A “bind-washout” process for the manufacturing scale purification of antibody monomers from a recombinant antibody sample containing aggregates comprising the steps of:
a. choosing a resin suitable for manufacturing level purification;
b. determining a pI value for the antibody monomer to be purified;
c. determining a pH value and a salt concentration to be used in the manufacturing level purification based on the pl value of step (b), wherein the aggregates bind to the resin and wherein the antibody monomers interact weakly with the resin; and
d. loading the recombinant antibody sample onto the chosen resin and washing the antibody monomers from the resin with a single buffer resulting in manufacturing level purification of the antibody monomers.

2. A “bind-elute” process for the manufacturing scale purification of antibody monomers from a recombinant antibody sample containing aggregates comprising the steps of:
a. choosing a resin suitable for manufacturing level purification;
b. determining a pH value and a salt concentration to be used in the manufacturing level purification such that the antibody monomers and the aggregates bind to the resin;
c. loading the recombinant antibody sample onto the chosen resin; and
d. eluting the antibody monomers from the resin using a step gradient.

3. The process according to claim 1 or 2, wherein the resin is an anion exchange resin.

4. The process according to claim 3, wherein the anion exchange resin is Q-SEPHAROSE FF.

5. The process of claim 2, wherein a pI value of the antibody monomers is determined prior to step (b).

6. The process according to claim 1 or claim 5, wherein the pH of the buffer is greater than the pI of the antibody.

7. The process according to claim 1 or claim 2, wherein the buffer comprises a salt concentration ranging from about 1 mM to about 500 mM.

8. The process according to claim 1 or claim 2, wherein the salt concentration ranges from about 25 mM to about 250 mM.

9. The process of claim 1 or claim 2, wherein the salt comprises NaCl.

10. The process according to claim 1 or claim 2, wherein the aggregate present in the sample after anion exchange is less than 0.5%.

11. The process according to claim 1 or claim 2, wherein the aggregate present in the sample after anion exchange is less than 0.1%.

12. The process according to claim 1 or claim 2, wherein about greater than 70% of the antibody is recovered.

13. The process according to claim 1 or claim 2, wherein about 90% of the antibody is recovered.
